(19) United States
(12) Patent Application Publication
(10) Pub. No.: US 2007/0281031 A1
(43) Pub. Date: Dec. 6, 2007

(54) Microparticles and methods for production thereof

(76) Inventor: Guohan Yang, Mansfield, MA (US)

Correspondence Address: BAXTER HEALTHCARE CORPORATION, ONE BAXTER PARKWAY, DF2-2E, DEERFIELD, IL, 60015, US

(21) Appl. No.: 11/421,575

(22) Filed: Jun. 1, 2006

(51) Int. Cl. A61K 38/09; A61K 9/14

(52) U.S. Cl. 424/489; 514/15

(57) ABSTRACT

An active agent and a non-polymeric polyanionic compound are allowed to be associated with a microparticle containing an anionic macromolecule and an anionic polymer in the presence of a crosslink activator. In a non-limiting example, the non-polymeric polyanionic compound is a polyanionic amino acid such as aspartic acid or glutamic acid. The microparticle can be used for modified release of the active agent in vitro and/or in vivo.

### TECHNICAL FIELD

The present disclosure generally relates to microparticles and to methods for the production of microparticles. Microparticles have an average geometric particle size (sometimes referred to as diameter) of less than 1 millimeter. Microparticles have been used in many different applications, primarily separations, diagnostics, and drug delivery.

### SUMMARY

There are several aspects of the present disclosure. In one aspect, the disclosure provides for microparticle compositions and methods for making the microparticles including at least one active agent and at least one non-polymeric polyanionic compound. In another aspect, the present disclosure provides for microparticle compositions and methods for making the microparticles including at least one active agent having oligopeptide segments and/or being free of primary amine groups and/or carboxyl groups. In an aspect of the microparticles, the active agent can include at least one nucleophilic group, which may be an hydroxyl group.

In a further aspect, the present disclosure provides for microparticle compositions and methods for making the compositions that include at least one active agent and at least one non-polymeric polyanionic compound that comprises at least one of diacids, triacids, polyanionic amino acids, derivatives thereof, and combinations of two or more thereof.

In another aspect of the present disclosure, microparticle compositions are provided by a method that includes combining a preformed microparticle, at least one active agent, and at least one non-polymeric polyanionic compound, and exposing the combination to at least one crosslink activator. The method is capable of preparing a microparticle, in which the preformed microparticle is in association with the at least one active agent and the at least one non-polymeric polyanionic compound. In one example, the at least one active agent is in association with the at least one non-polymeric polyanionic compound.

Another aspect of this disclosure provides microparticle compositions including at least one anionic macromolecule, at least on anionic polymer, at least one active agent and at least one non-polymeric polyanionic compound. In a further aspect, the anionic macromolecule and the anionic polymer are homogeneously distributed with each other.

Other aspects, objects and advantages of the present disclosure will be understood from the following description according to the illustrated embodiments, specifically including stated and unstated combinations of the various features which are described herein, relevant information concerning which is shown in the accompanying drawings.

### BRIEF DESCRIPTION OF THE DRAWINGS

In the course of this description, reference will be made to the attached drawings, wherein:

FIG. 1 is an illustration showing possible, but non-limiting, reaction schemes of different covalent associations among leuprolide, aspartic acid, and human serum albumin (HSA) in microparticles.

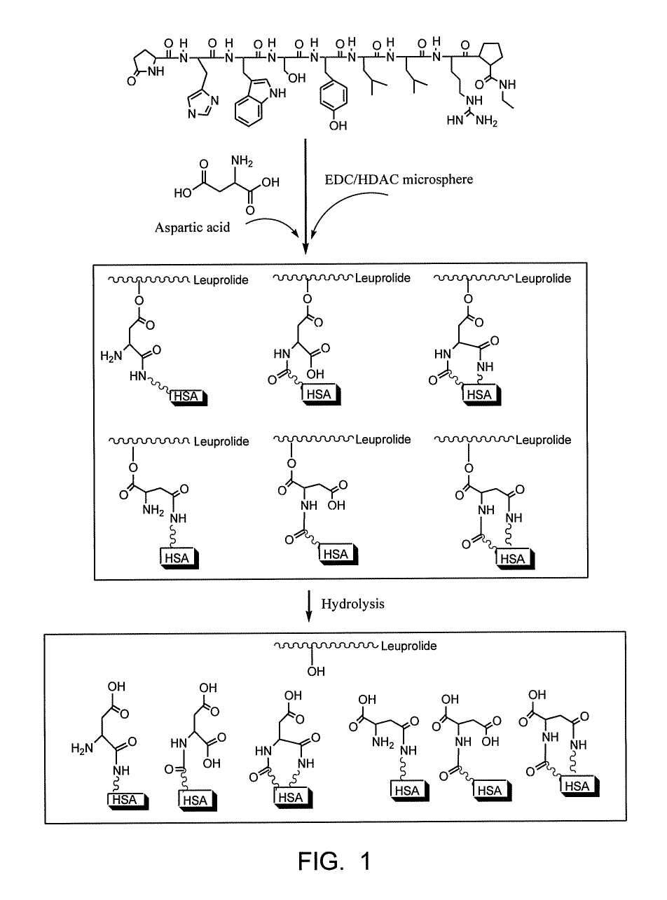

FIG. 2 is a plot showing the release kinetics of leuprolide from microparticles according to the disclosure of Example 1.

FIG. 3 is a plot showing the release kinetics of leuprolide from microparticles according to the disclosure of Example 3.

FIG. 4 is a plot showing the release kinetics of leuprolide from microparticles according to the disclosure of Example 4.

### DETAILED DESCRIPTION

Microparticles can be used to provide a preparation capable of modified release (such as controlled release) of at least one active agent that is incorporated in or otherwise associated with the microparticles. To be an effective delivery vehicle, the microparticles can be designed such that the at least one associated active agent is capable of displaying a desired release profile. The preparation would substantially retain, if not observably enhance, the effectiveness of the at least one active agent.

Without being limited thereto, the present disclosure is directed in general to microparticles containing at least one macromolecule (such as aqueous-soluble macromolecules and anionic macromolecules, like human serum albumin), at least one polymer (such as aqueous-soluble polymers and anionic polymers, like dextran sulfate), at least one active agent (such as proteinaceous compounds and nucleic acids, like leuprolide and goserelin and salts thereof), and at least one non-polymeric polyanionic compound (such as diacids and polyanionic amino acids, like aspartic acid and glutamic acid, anhydrides thereof, analogs thereof, and salts thereof). In one example, the non-polymeric polyanionic compound has two or more of the same or different anionic and/or negatively ionizable functional groups (such as carboxyl groups) and, optionally, one or more of the same or different cationic and/or positively ionizable functional groups (such as one or more amine groups). Non-limiting examples of non-polymeric polyanionic compounds include diacids (such as dicarboxylic acids, like succinic acid, oxalic acid, malonic acid, glutaric acid, adipic acid, pimelic acid, suberic acid, maleic acid, fumaric acid); triacids (such as tricarboxylic acids, like citric acid, tricarballylic acid, trimellitic acid, carboxymethyloxysuccinic acid, nitrilotriacetic acid); tetracids (such as tetracarboxylic acids, like ethylenediamine tetraacetic acid, 1,2,3,4-butanetetracarboxylic. acid); polyanionic amino acids (typically having more acid groups than amino groups), for example, amino diacids (such as dicarboxylic amino acids, like aspartic acid, glutamic acid, kainic acid, β-hydroxyaspartic acid, β-hydroxyglutamic acid, β-methylaspartic acid, β-methylglutamic acid, 2-aminoadipic acid), amino triacids (such as tricarboxylic amino acids, like carboxyglutamate, aconitic acid, domoic acid), diamino triacids, as well as anhydrides thereof, analogs thereof, and salts thereof (e.g., salts having one or more cations, such as monovalent metal cations like Na⁺ and K⁺, divalent metal cations like Ca²⁺, Mg²⁺, Zn²⁺, Fe²⁺, Cu²⁺, other polyvalent cations like Al³⁺ and Fe³⁺, as well as organic cations like tetraalkyl ammonium where the alkyl groups are independently chosen from C₁ to C₆ linear or branched alkyl groups, such as methyl, ethyl, propyl, isopropyl, butyl, isobutyl, sec-butyl, t-butyl, amyl, isoamyl, sec-amyl, t-amyl, hexyl, isohexyl, sec-hexyl, and t-hexyl groups). In one non-limiting example, the non-polymeric polyanionic compound is aqueous-soluble, such as being water-soluble. In another non-limiting example, the non-polymeric polyanionic compound is not a fatty acid, which includes fatty monoacids, fatty diacids, and other fatty polyacids. In a further non-limiting example, the at least one active agent is protonatable or protonated (partially or fully, for example, as in aqueous solution or in salt form), such as the agonists, antagonists, and analogs of LHRH and their salts disclosed herein. In a further non-limiting example, the microparticle of the present disclosure is free of lipids.

In another example, the active agent is free of functional amine groups and free of functional carboxyl groups, and has one or more of the same or different nucleophilic groups (such as hydroxyl groups). In a further example, the active agent contains one or more of serine, threonine, and tyrosine residues. In a further example, the active agent contains one or more of the same or different oligopeptide segments (such as tripeptide segments), each of such segments having a general structure Z₁-Z₂-Z₃, where Z₁ is histidine residue, Z₂ is different from Z₁ and Z₃, Z₂ being a single amino acid residue or a segment containing two or more amino acid residues, and Z₃ is serine residue or threonine residue. Non-limiting examples of suitable active agents include leuprolide, goserelin, buserelin, gonadorelin, histrelin, nafarelin, deslorelin, fertirelin, triptorelin, agonists thereof, antagonists thereof, analogs thereof, salts thereof (e.g., acetate, trifluoroacetate, hydrazide, amide, and hydrochloride), and combinations of two or more thereof. At least some of these active agents are cationic.

In a further example, a non-limiting method for preparing the microparticles involves providing preformed microparticles as substrate, exposing the preformed microparticles to at least one active agent and at least one non-polymeric polyanionic compound (simultaneously, sequentially, or separately), and forming the microparticles each containing the preformed microparticle, the at least one active agent, and the at least one non-polymeric polyanionic compound. Each of the resulting microparticles typically contains at least one of the preformed microparticles. The preformed microparticle can be associated (covalently, electrostatically, and/or otherwise) with the at least one active agent and/or the at least one non-polymeric polyanionic compound during and/or following the formation of the microparticle. The at least one non-polymeric polyanionic compound can be associated (covalently, electrostatically, and/or otherwise) with the at least one active agent prior to, during, and/or following the formation of the microparticle. In one example, the preformed microparticles are provided in the form of a suspension in a non-solvent medium, such as an aqueous solution. The preformed microparticles can be formed from two or more materials, such as a homogeneous mixture of at least one macromolecule (like human serum albumin) and at least one polymer (like dextran sulfate). The exposing process can be carried out in the presence of at least one crosslink activator (such as carbodiimides and salts thereof, like 1-ethyl-3-(3-dimethylaminopropyl)carbodiimide hydrochloride, 1-ethyl-3-(4-azonia-4,4-dimethylpentyl)carbodiimide, 1-cyclohexyl-3-(2-morpholinoethyl)carbodiimide metho-p-toluenesulfonate, and combinations of two or more thereof). In one example, the at least one crosslink activator is mixed into the suspension of preformed microparticles following the addition of the at least one non-polymeric polyanionic compound, at a temperature between 10° C. and 60° C. and/or a pH of 6.5 or lower. The resulting microparticles can be used directly (such as in a suspension form) without further processing or, optionally, processed into a useful form (such as a suspension or a dry powder) through, for example, centrifugal washing, filtration, diafiltration, dialysis, and/or lyophilization.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Thus, for example, the reference to a particular microparticle is a reference to one such microparticle or a plurality of such microparticles, including equivalents thereof known to one skilled in the art. Also, as used herein and in the claims, the terms “at least one” and “one or more” have the same meaning and include one, two, three or more. The following terms, unless otherwise indicated, shall be understood to have the following meanings when used in the context of the present disclosure.

“Active agent” refers to naturally occurring, synthetic, or semi-synthetic materials (e.g., compounds, fermentates, extracts, cellular structures) capable of eliciting, directly or indirectly, one or more physical, chemical, and/or biological effects, in vitro and/or in vivo. The active agent can be capable of preventing, alleviating, treating, and/or curing abnormal and/or pathological conditions of a living body, such as by destroying a parasitic organism, or by limiting the effect of a disease or abnormality by materially altering the physiology of the host or parasite. The active agent can be capable of maintaining, increasing, decreasing, limiting, or destroying a physiologic body function. The active agent can be capable of diagnosing a physiological condition or state by an in vitro and/or in vivo test. The active agent can be capable of controlling or protecting an environment or living body by attracting, disabling, inhibiting, killing, modifying, repelling and/or retarding an animal or microorganism. The active agent can be capable of otherwise treating (such as deodorizing, protecting, adorning, grooming) a body. Depending on the effect and/or its application, the active agent can further be referred to as a bioactive agent, a pharmaceutical agent (such as a prophylactic agent, a therapeutic agent), a diagnostic agent, a nutritional supplement, and/or a cosmetic agent, and includes, without limitation, prodrugs, affinity molecules, synthetic organic molecules, polymers, molecules with a molecular weight of 2 kD or less (such as 1.5 kD or less, or 1 kD or less), macromolecules (such as those having a molecular weight of 2 kD or greater, or 5 kD or greater), proteinaceous compounds, peptides, vitamins, steroids, steroid analogs, lipids, nucleic acids, carbohydrates, precursors thereof, and derivatives thereof. Active agents can be ionic or non-ionic, can be neutral, positively charged, negatively charged, or zwitterionic, and can be used singly or in combination of two or more thereof. Active agents can be water-insoluble or water-soluble. Active agents can have an isoelectric point of 7.0 or greater, or less than 7.0.

“Non-polymeric polyanionic compound” refers to compounds other than polymers that have two or more deprotonatable groups such as acid groups, and their corresponding anhydrides, analogs, and salts. In one non-limiting example, the non-polymeric polyanionic compound is aqueous-soluble, such as being water-soluble. In another non-limiting example, the non-polymeric polyanionic compound is not a fatty acid, which includes fatty monoacids, fatty diacids, and other fatty polyacids.

“Microparticle” refers to a particulate that is solid (including substantially solid or semi-solid, but excluding gel, liquid and gas), having an average geometric particle size (sometimes referred to as diameter) of less than 1 mm, such as 200 microns or less, 100 microns or less, or 10 microns or less. Average geometric particle sizes can range between values such as these and 0.01 microns or greater, such as 0.1 microns or greater or 0.5 microns or greater. In one example, the average geometric particle size can range from 0.5 microns to 5 microns. Average geometric particle size can be measured by dynamic light scattering methods (such as photocorrelation spectroscopy, laser diffraction, low-angle laser light scattering (LALLS), medium-angle laser light scattering (MALLS)), light obscuration methods (such as Coulter analysis method), or other methods (such as rheology, light or electron microscopy). Particles for pulmonary delivery will have an aerodynamic particle size determined by time of flight measurements or Andersen Cascade Impactor measurements. Microparticles can have a spherical shape (sometimes referred to as microspheres) and/or can be encapsulated (sometimes referred to as microcapsules). Certain microparticles can have one or more internal voids and/or cavities. Other microparticles can be free of such voids or cavities. Microparticles can be porous or non-porous. Microparticles can be formed from, in part or in whole, one or more non-limiting materials, such as the active agents, carriers, polymers, and/or stabilizing agents disclosed herein.

“Peptides” refer to natural, synthetic, or semi-synthetic compounds formed at least in part from two or more of the same or different amino acids and/or imino acids. Non-limiting examples of peptides include oligopeptides (such as those having less than 50 amino/imino acid monomer units, including dipeptides and tripeptides and the like), polypeptides, proteinaceous compounds as defined herein, as well as precursors and derivatives thereof (e.g., glycosylated, hyperglycosylated, PEGylated, FITC-labeled, salts thereof). Peptides can be used singly or in combination of two or more thereof. Peptides can be neutral, positively charged, negatively charged, or zwitterionic, and can be used singly or in combination of two or more thereof.

“Proteinaceous compounds” refer to natural, synthetic, semi-synthetic, or recombinant compounds of or related structurally and/or functionally to proteins and compounds containing or consisting essentially of α-amino acids covalently associated through peptide linkages, and include precursors, variants, analogs, derivatives, agonists, antagonists, as well as combinations of two or more thereof. Naturally occurring proteinaceous compounds include those of whatever species, for example, human, bovine, porcine, canine, or feline. The amino acid portion of the proteinaceous compounds, or a precursor thereto, can be made by solid-phase synthetic chemistry, purification from natural sources, recombinant DNA technologies, synthetic organic techniques such as alkylation and acylation, and combinations of two or more thereof.

“Precursor” refers to any material or substance capable of being converted to a desired material or substance, such as through a chemical and/or biochemical reaction or pathway, like anchoring one or more precursor moieties to a material. Non-limiting precursor moieties include maleimide groups, disulfide groups (e.g., ortho-pyridyl disulfide), vinylsulfone groups, azide groups, and α-iodo acetyl groups. Precursors of the proteinaceous compounds further include reduced (—SH) forms and S-protected forms, for example, S-sulfonates of different proteinaceous compounds such as hormones.

“Analog” refers to a compound having a chemically modified form of a principal compound or class thereof, which maintains the pharmaceutical and/or pharmacological activities characteristic of the principal compound or class. Analogs of proteinaceous compounds and their precursors include, for example, a molecule having one or more amino acid substitutions, deletions, inversions, or additions compared with the principal compound.

“Derivative” refers to any material or substance formed from a parent material or substance, such as through one or more chemical and/or biochemical reactions or pathways. Non-limiting examples of derivatives include glycosylated, hyperglycosylated, PEGylated, FITC-labelled, protected with protecting groups (e.g., benzyl for alcohol or thiol, t-butoxycarbonyl for amine), as well as salts, esters, amides, conjugates, complexes, manufacturing related compounds, and metabolites thereof. Salts can be organic or inorganic, with cations that are monovalent or polyvalent, metallic, organic, or organometallic, and anions that are monovalent or polyvalent, organic, inorganic, or organometallic. Salts that are pharmaceutically acceptable include, without limitation, mineral or organic acid salts of basic residues (e.g., amines), alkali or organic salts of acidic residues (e.g., carboxylic acids), and the like, such as conventional non-toxic salts or the quaternary ammonium salts of the parent compound formed from non-toxic inorganic acids (e.g., hydrochloric, hydrobromic, sulfuric, sulfamic, phosphoric, nitric) and organic acids (e.g., acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic). Derivatives of proteinaceous compounds and their precursors include, for example, a molecule having the amino acid sequence of the principal compound or its analog, but additionally having chemical modification of one or more of its amino acid side groups, alpha-carbon atoms, terminal amino groups, and/or terminal carboxylic acid groups.

The proteinaceous compounds further include variants of the principal compounds and their precursors, for example, structures which have been modified to lengthen and/or shorten the amino acid sequence, for example, the 20K variant of hormones, methionyl hormones, and the like. Non-limiting proteinaceous compounds include globular proteins (e.g., albumins, globulins, histones), fibrous proteins (e.g., collagens, elastins, keratins), compound proteins (including those containing one or more non-peptide component, e.g., glycoproteins, nucleoproteins, mucoproteins, lipoproteins, metalloproteins), therapeutic proteins, fusion proteins, receptors, antigens (such as synthetic or recombinant antigens), viral surface proteins, hormones and hormone analogs, antibodies (such as monoclonal or polyclonal antibodies), enzymes, Fab fragments, cyclic peptides, linear peptides, as well as precursors thereof, analogs thereof, derivatives thereof, variants thereof, fragments thereof, agonists thereof, antagonists thereof, and combinations of two or more thereof. Non-limiting therapeutic proteinaceous compounds include bone morphogenic proteins, drug resistance proteins, toxoids, erythropoietins, proteins of the blood clotting cascade (e.g., Factor VII, Factor VIII, Factor IX, et al.), subtilisin, ovalbumin, alpha-1-antitrypsin (AAT), DNase, superoxide dismutase (SOD), lysozyme, ribonuclease, hyaluronidase, collagenase, growth hormones such as human growth hormone (hGH), erythropoietin, insulin and its analogs, insulin-like growth factors and their analogs, interferons, glatiramer, granulocyte-macrophage colony-stimulating factor, granulocyte colony-stimulating factor, desmopressin, leutinizing hormone release hormone (LHRH) and its agonists and analogs (e.g., leuprolide, goserelin, buserelin, gonadorelin, histrelin, nafarelin, deslorelin, fertirelin, triptorelin), LHRH antagonists, vasopressin, cyclosporine, calcitonin, parathyroid hormone, parathyroid hormone peptides, and glucogen-like peptides and their analogs.

“Nucleic acids” refer to natural, synthetic, semi-synthetic, or recombinant compounds formed at least in part from two or more of the same or different nucleotides, and can be single-stranded or double-stranded. Non-limiting examples of nucleic acids include oligonucleotides (such as those having 20 or less base pairs, e.g., sense, anti-sense, or missense), aptamers, polynucleotides (e.g., sense, anti-sense, or missense), DNA (e.g., sense, anti-sense, or missense), RNA (e.g., sense, anti-sense, or missense), siRNA, nucleotide acid constructs, single-stranded or double-stranded segments thereof, as well as precursors and derivatives thereof (e.g., glycosylated, hyperglycosylated, PEGylated, FITC-labeled, nucleosides, salts thereof). Nucleic acids can be neutral, positively charged, negatively charged, or zwitterionic, and can be used singly or in combination of two or more thereof.

“Carbohydrates” refer to natural, synthetic, or semi-synthetic compounds formed at least in part from monomeric sugar units. Non-limiting carbohydrates include polysaccharides, sugars, starches, and celluloses, such as carboxymethylcellulose, dextrans, hetastarch, cyclodextrins, alginates, chitosans, chondroitins, heparins, as well as precursors and derivatives thereof (e.g., glycosylated, hyperglycosylated, PEGylated, FITC-labeled, salts thereof). Carbohydrates can be ionic or non-ionic, can be neutral, positively charged, negatively charged, or zwitterionic, and can be used singly or in combination of two or more thereof.

“Lipids” refer to natural, synthetic, or semi-synthetic compounds that are generally amphiphilic. The lipids typically comprise a hydrophilic component and a hydrophobic component. Non-limiting examples include fatty acids, neutral fats, phosphatides, oils, glycolipids, surfactants, aliphatic alcohols, waxes, terpenes and steroids. Lipids can be ionic or non-ionic, can be neutral, positively charged, negatively charged, or zwitterionic, and can be used singly or in combination of two or more thereof.

“Stabilizing,” used especially in conjunction with an agent (e.g., compound), a process, or a condition, refers to the capability of such agent, process or condition to, at least in part, form the microparticles (or a composition or formulation or kit containing such microparticles), facilitate the formation thereof, and/or enhance the stability thereof (e.g., the maintenance of a relatively balanced condition, like increased resistance against destruction, decomposition, degradation, and the like). Non-limiting stabilizing processes or conditions include thermal input/output (e.g., heating, cooling), electromagnetic irradiation (e.g., gamma rays, X rays, UV, visible light, actinic, infrared, microwaves, radio waves), high-energy particle irradiation (e.g., electron beams, nuclear), and ultrasound irradiation. Non-limiting stabilizing agents include lipids, proteins, polymers, carbohydrates, surfactants, salts (e.g., organic, inorganic, with cations that are monovalent or polyvalent, metallic, organic, or organometallic, and anions that are monovalent or polyvalent, organic, inorganic, or organometallic), as well as certain of the carriers, the active agents, the crosslinkers, and the co-agents (such as the non-polymeric polyanionic compounds) disclosed herein. The stabilizing agents can be ionic or non-ionic, can be neutral, positively charged, negatively charged, or zwitterionic, and can be used singly or in combination of two or more thereof.

“Macromolecule” refers to a material capable of providing a three-dimensional (e.g., tertiary and/or quaternary) structure, and includes carriers and certain active agents of the present disclosure. Non-limiting macromolecules used to form the microparticles include, inter alia, polymers, copolymers, proteins (e.g., enzymes, recombinant proteins, albumins like human serum albumin), peptides, lipids, carbohydrates, polysaccharides, nucleic acids, vectors (e.g., virus, viral particles), complexes and conjugates thereof (e.g., by covalent and/or non-covalent associations, between two macromolecules like carbohydrate-protein conjugates, between an active agent and a macromolecule like hapten-protein conjugates, the active agent can or can not be capable of having a tertiary and/or quaternary structure), and combinations of two or more thereof. Macromolecules typically have a molecular weight of 1,500 or greater. Macromolecules can be neutral, positively charged, negatively charged, or zwitterionic, and can be used singly or in combination of two or more thereof.

“Spherical” refers to a geometric shape that is at least “substantially spherical.” “Substantially spherical” means that the ratio of the longest length (i.e., one between two points on the perimeter and passes the geometric center of the shape) to the shortest length on any cross-section that passes through the geometric center is about 1.5 or less, such as about 1.33 or less, or about 1.25 or less. Spherical does not require a line of symmetry. Further, the microparticles can have surface texturing (such as continuous or discrete lines, islands, lattice, indentations, channel openings, protuberances that are small in scale when compared to the overall size of the microparticles) and still be spherical. Surface contact therebetween is minimized in microparticles that are spherical, which minimizes the undesirable agglomeration of the microparticles. In comparison, microparticles that are crystals or flakes typically display observable agglomeration through ionic and/or non-ionic interactions at relatively large flat surfaces.

“Monodisperse size distribution” refers to a microparticle size distribution in which the ratio of the volume diameter of the 90^{th} percentile (i.e., the average particle size of the largest 10% of the microparticles) to the volume diameter of the 10^{th} percentile (i.e., the average particle size of the smallest 10% of the microparticles) is about 5 or less, such as about 3 or less, about 2 or less, or about 1.5 to 1. Consequently, “polydisperse size distribution” refers to one where the diameter ratio described above is greater than 5, such as 8 or greater, or 10 or greater. In microparticles having a polydisperse size distribution, smaller microparticles can fill in the gaps between larger microparticles, thus possibly displaying large contact surfaces and observable agglomeration there between. A Geometric Standard Deviation (GSD) of 2.5 or less, such as 1.8 or less, can also be used to indicate a monodisperse size distribution. Calculation of GSD is known and understood to one skilled in the art.

“Amorphous” refers to materials and constructions that are “substantially amorphous,” such as microparticles having multiple non-crystalline domains (or lacking crystallinity altogether) or otherwise non-crystalline. Substantially amorphous microparticles of the present disclosure are generally random solid particulates in which crystalline lattices constitute less than 50% by volume and/or weight of the microparticles, or are absent, and include semi-crystalline microparticles and non-crystalline microparticles as understood by one skilled in the art.

“Solid” refers to a state that includes at least substantially solid and/or semi-solid, but excludes gel, liquid, and gas.

“Preformed microparticle” refers to a microparticle fabricated using one or more non-limiting methods, such as those known to one skilled in the art, without surface modification as described herein, having or capable of having on its outer surface a net surface electric charge that is positive, negative, or neutral. A preformed microparticle is also referred to herein as “core microparticle” or “core.” The preformed or core microparticle typically comprises one or more active agents and, optionally, one or more carriers, which, independently, can be compartmentalized in a portion of the preformed or core microparticle, or be distributed substantially homogeneously throughout the preformed microparticles. The net surface charge, typically being non-zero, can be contributed primarily, or at least substantially, by the active agent(s) and/or the optional carrier compounds.

“Carrier” refers to a compound, typically a macromolecule, having a primary function to provide a three-dimensional structure (including tertiary and/or quaternary structure). The carrier can be unassociated or associated with the active agent (such as conjugates or complexes thereof) in forming microparticles as described above. The carrier can further provide other functions, such as being an active agent, modify release profile of the active agent from the microparticle, and/or impart one or more particular properties to the microparticle (such as contribute at least in part to the net surface charge). In one example, the carrier is a protein (such as albumins, like human serum albumin) having a molecular weight of 1500 Daltons or greater.

“Polymer” or “polymeric” refers to a natural, synthetic, or semi-synthetic molecule having in a main chain or ring structure two or more repeating monomer units. Polymers broadly include dimers, trimers, tetramers, oligomers, higher molecular weight polymer, adducts, homopolymers, random copolymers, pseudo-copolymers, statistical copolymers, alternating copolymers, periodic copolymer, bipolymers, terpolymers, quaterpolymers, other forms of copolymers, substituted derivatives thereof, and mixtures thereof, and narrowly refer to molecules having 10 or more repeating monomer units. Polymers can be linear, branched, block, graft, monodisperse, polydisperse, regular, irregular, tactic, isotactic, syndiotactic, stereoregular, atactic, stereoblock, single-strand, double-strand, star, comb, dendritic, and/or ionomeric, can be ionic or non-ionic, can be neutral, positively charged, negatively charged, or zwitterionic, and can be used singly or in combination of two or more thereof.

“Suspension” or “dispersion” refers to a mixture, typically finely divided, of two or more phases (e.g., solid, liquid, gas), such as solid in liquid, liquid in liquid, gas in liquid, solid in solid, solid in gas, liquid in gas, and the like. The suspension or dispersion can remain stable for extended periods of time (e.g., minutes, hours, days, weeks, months, years).

“Resuspending” refers to changing microparticles from a non-flowable (e.g., solid) state to a flowable (e.g., liquid) state by adding a flowable medium (e.g., a liquid), while retaining most or all of the characteristics of the microparticles. The liquid can be, for example, aqueous, aqueous miscible, or organic.

“Ambient temperature” refers to a temperature of around room temperature, typically in a range of about 20° C. to about 40° C.

“Therapeutic” refers to any pharmaceutic, drug, prophylactic agent, contrast agent, or dye useful in the treatment (including prevention, diagnosis, alleviation, suppression, remission, or cure) of a malady, affliction, disease or injury in a subject. Therapeutically useful peptides and nucleic acids can be included within the meaning of the term “pharmaceutic” or “drug.”

“Cross-link,” “cross-linked” and “cross-linking” generally refer to the linking of two or more materials and/or substances, including any of those disclosed herein, through one or more covalent and/or non-covalent (e.g., ionic) associations. Cross-linking can be effected naturally (e.g., disulfide bonds of cystine residues) or through synthetic or semi-synthetic routes, for example, optionally in the presence of one or more cross-linkers (i.e., a molecule X by itself capable of reacting with two or more materials/substances Y and Z to form a cross-link product Y-X-Z, where the associations of Y-X and X-Z are independently covalent and/or non-covalent), initiators (i.e., a molecule by itself capable of providing reactive species like free radicals for the cross-link reaction, e.g., thermally decomposable initiators like organic peroxides, azo initiators, and carbon-carbon initiators, actinically decomposable initiators like photoinitiators of various wavelengths), activators (i.e., a molecule A capable of reacting with a first material/substance Y to form an activated intermediate [A-Y], which in turn reacts with a second material/substance Z to form a cross-link product Y-Z, while A is chemically altered or consumed during the process), catalysts (i.e., a molecule capable of modifying the kinetics of the cross-link reaction without being chemically modified during the process), co-agents (i.e., a molecule, typically a non-polymeric polyanionic compound, when co-present with one or more of the initiators, activators, and/or catalysts, is capable of modifying the kinetics of the cross-link reaction and/or being associated with resulting product such as the microparticles), and/or energy sources (e.g., heating; cooling; high-energy radiations like electromagnetic, e-beam, and nuclear; acoustic radiations like ultrasonic; etc.).

“Covalent association” refers to an intermolecular interaction (e.g., a bond) between two or more individual molecules that involves the sharing of electrons in the bonding orbitals of two atoms.

“Non-covalent association” refers to an intermolecular interaction between two or more individual molecules without involving a covalent bond. Intermolecular interaction depends on, for example, polarity, electric charge, and/or other characteristics of the individual molecules, and includes, without limitation, electrostatic (e.g., ionic) interactions, dipole-dipole interactions, van der Waals' forces, and combinations of two or more thereof.

“In association with” and “associated with” refer in general to the one or more interactions between, and/or incorporation of, different materials (typically those that are part of the microparticles), one or more of such materials and one or more structures (or portions thereof) of the microparticles, and different structures (or portions thereof) of the microparticles. The materials of the microparticles include, without limitation, ions such as monovalent and polyvalent ions disclosed herein, as well as compounds such as active agents, stabilizing agents, cross-link agents, charged or uncharged compounds, the various polymers disclosed herein, and combinations of two or more thereof. The structures of the microparticles and portions thereof include, without limitation, core, core microparticle, preformed microparticle, monolayer, intermediate microparticle, surface-modified microparticle, portions of such structures (such as outer surfaces, inner surfaces), domains between such structures and portions thereof, and combinations of two or more thereof. Various associations, being reversible or irreversible, migratory or non-migratory, can be present singly or in combination of two or more thereof. Non-limiting associations include, without limitation, covalent and/or non-covalent associations (e.g., covalent bonding, ionic interactions, electrostatic interactions, dipole-dipole interactions, hydrogen bonding, van der Waals' forces, cross-linking, and/or any other interactions), encapsulation in layer/membrane, compartmentalization in center or vesicles or between two layers/membranes, homogeneous integration throughout the microparticle or in a portion thereof (e.g., containment in, adhesion to, and/or affixation to center or layer or vesicle or an inner and/or outer surface thereof; interspersion, conjugations, and/or complexation between different materials).

“Controlled release” refers to a predetermined in vivo and/or in vitro release (e.g., dissolution) profile of an active agent, as compared to the release profile of the active agent in its native form. The active agent is associated with a microparticle or a composition or formulation containing such a microparticle, as disclosed herein, such that one or more aspects of its release kinetics (e.g., initial burst, quantity and/or rate over a specified time period or phase, cumulative quantity over a specific time period, length of time for total release, pattern and/or profile, etc.) are increased, decreased, shortened, prolonged, and/or otherwise modified as desired. Non-limiting examples of controlled release include immediate/instant release (i.e., initial burst or rapid release), extended release, sustained release, prolonged release, delayed release, modified release, and/or targeted release, occurring individually, in combination of two or more thereof, or in the absence of one or more thereof (e.g. extended or sustained release in the absence of an initial burst).

“Extended release” refers to the release of an active agent in association with a microparticle or a composition or formulation containing such a microparticle, as disclosed herein, over a time period longer than the free aqueous diffusion period of the active agent in its native form. The extended release period can be hours (e.g., at least about 1, 2, 5, or 10 hours), days (e.g., at least about 1, 2, 3, 4, 5, 6, 7, 8, 10, 15, 20, 30, 40, 45, 60, or 90 days), weeks (at least about 1, 2, 3, 4, 5, 6, 10, 15, 20, 30, 40, or 50 weeks), months (at least about 1, 2, 3, 4, 6, 9, or 12 months), about 1 or more years, or a range between any two of the time periods. The pattern of an extended release can be continuous, periodic, sporadic, or a combination thereof.

“Sustained release” refers to an extended release of an active agent such that a functionally significant level of the active agent (i.e., a level capable of bringing about the desired function of the active agent) is present at any time point of the extended release period, typically with a continuous and/or uniform release pattern. Non-limiting examples of sustained release profiles include those, when displayed in a plot of release time (x-axis) versus cumulative release (y-axis), showing at least one upward segment that is linear, step-wise, zig-zagging, curved, and/or wavy, over a time period of 1 hour or longer.

Other than in the operating Examples, or unless otherwise expressly specified, all of the numerical ranges, amounts, values and percentages such as those for quantities of materials, times, temperatures, reaction conditions, ratios of amounts, values for molecular weight (whether number average molecular weight Mₙ or weight average molecular weight M_{w}), and others disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values can be used.

“Formed from” and “formed of” denote open language. As such, it is intended that a composition “formed from” or “formed of” a list of recited components be a composition comprising at least these recited components, and can further include other non-recited components during formulation of the composition.

Examples provided herein, including those following “such as” and “e.g.,” are considered as illustrative only of various aspects of the present disclosure and embodiments thereof, without being specifically limited thereto. Any suitable equivalents, alternatives, and modifications thereof (including materials, substances, constructions, compositions, formulations, means, methods, conditions, etc.) known and/or available to one skilled in the art can be used or carried out in place of or in combination with those disclosed herein, and are considered to fall within the scope of the present disclosure. Therefore, specific details disclosed herein are not to be interpreted as limiting, but merely as a basis for the claims and as a representative basis for teaching one skilled in the art to variously employ aspects of the present disclosure in virtually any appropriate manner.

Microparticles are a form of delivering, in vivo and/or in vitro, active agents with controlled release profiles, and are useful for a wide variety of therapeutic, pharmaceutical, diagnostic, medical, medicinal, cosmetic, nutritional, biocidic, separational, industrial, commercial, and research purposes, such as drug delivery, vaccination, gene therapy and histopathological or in vivo tissue or tumor imaging. Microparticles can be suitable for oral, parenteral, mucosal; ophthalmic; intravenous, subcutaneous, intra-articular, intramuscular, pulmonary, and/or topical administrations to a subject. Intravenous administration includes catheterization and angioplasty. Non-limiting microparticles, materials and methods for fabricating microparticles, compositions and formulations containing microparticles, and utilities of microparticles, compositions, and formulations include those disclosed herein and those described in U.S. Pat. Nos. 6,090,925, 6,268,053, and 6,458,387, the disclosure of which are herein incorporated by reference in their entirety. Microparticles can have a generally uniform size distribution, such as a monodisperse size distribution, and a generally uniform shape, such as being substantially spherical. One or more characteristics of the microparticles can be adjusted during fabrication by manipulating one or more variables such as, but are not limited to, selection of ingredients or combination thereof, concentrations of different ingredients, reaction temperature, reaction time, pH if reaction is taken place in aqueous solution.

In one example, the microparticles of the present disclosure, including the preformed microparticles, are free of internal voids or cavities, but can have, on their outermost surfaces, a plurality of randomly distributed channel openings. The channel openings can be similar or different in size and/or depth. As determined by, for example, gas adsorption technique using, for example, Brunauer-Emmett-Teller (BET) analysis, the channels typically have a diameter of 1,000 Angstroms or less, and can include macrochannels with a diameter of 500 Angstroms to 1,000 Angstroms, mesochannels with a diameter of 20 Angstroms to less than 500 Angstroms, microchannels with a diameter of 7 Angstroms to less than 20 Angstroms, and/or ultramicrochannels with a diameter of less than 7 Angstroms. The channel size distribution can be unimodal or bimodal. The surface channels, depending on their diameters, can be permeable to water and certain dissolved materials, allowing aqueous fluids to enter the microparticle and certain solubilized materials (e.g., active agent, polymer, carrier) of appropriate size to exit the microparticle.

In one example, the one or more materials (e.g., macromolecule, polymer, active agent, and/or stabilizing agent) that form the microparticle are substantially homogeneously distributed throughout the microparticle. The different materials can be intertwined and/or interspersed with one another, optionally covalently and/or non-covalently associated with one another. Although not wishing to be bound to any particular theory or mechanism, it is believed that when the inner portion (typically a matrix material) of the microparticle is water soluble and solubilized, ingredients forming the inner portion can at least in part diffuse out of the microparticle under appropriate conditions (e.g., in release medium such as body fluids, or a physiologically acceptable buffer under physiological conditions). The macromolecule ingredient(s) (such as carrier molecules) and/or the active agent(s) can comprise at least 40%, 50%, 60%, 70%, 80%, 90%, 95%, or 98%, and less than 100%, by weight and/or volume, of the microparticle, or in a range there between. Other ingredient(s) (e.g., polymers, stabilizing agents) can be present in a minor quantity of 30% or less, 20% or less, 10% or less, 5% or less, or 2% or less, and greater than 0%, by weight and/or volume of the microparticle, or in a range there between.

One or more of the various ingredients (e.g., macromolecule, polymer, active agent, and/or stabilizing agent) that form the microparticle can independently be labeled with a detectable label. Labels, methods of labeling the different ingredients (e.g., proteins, nucleic acids), and methods of detecting the labels include those known to those skilled in the art. Non-limiting labels include magnetic substances (e.g., metallic substances, metals), radiolabels (e.g., [32]P, [3]H, [14]C, [35]S, [125]I, [131]I), mass or nuclear magnetic resonance (NMR) labels (e.g., [13]C, [15]N, [19]O), dyes (e.g., ethidium bromide, acridine, propidium, intercalating dyes, 4′,6′-diamidino-2-phenylindole), chemiluminescent agents, bioluminescent agents, fluorogens (e.g., fluorescein and derivatives, phycoerythrin, allo-phycocyanin, phycocyanin, rhodamine, Texas Red), chromogens, biotin, antigens, affinity labels (a chemical group recognizable by specific antibodies), and combinations of two or more thereof. A [32]P label can be conjugated to a protein with a conjugating reagent or incorporated into the sequence of a nucleic acid molecule by nick-translation, end-labeling or incorporation of labeled nucleotide, a [3]H, [14]C, or [35]S label can be incorporated into a nucleotide sequence by incorporation of a labeled precursor or by chemical modification, whereas an [125]1 or [131]I label is generally incorporated into a nucleotide sequence by chemical modification. Methods of detecting a radiolabel include scintillation counting, gamma ray spectrometry, and autoradiography. Methods of detecting mass or NMR labels include mass spectrometry and magnetic resonance imaging. Methods of detecting dyes and fluorogens include spectrophotometry and fluorescence detection, respectively.

For example, one or more of the ingredients can be labeled with a chromogen (enzyme substrate) to provide an enzyme or affinity label. The ingredients can be biotinylated for use in a biotin-avidin reaction, optionally coupled to a second label such as an enzyme or a fluorogen. The ingredients can be labeled with peroxidase, alkaline phosphatase or other enzymes to give a chromogenic or fluorogenic reaction upon addition of substrate. Labeling can also be achieved by incorporating a labeled modified base, amino acid, or precursor. Bound antibody-antigen complex can be detected using enzyme-linked immunoassays (ELISA) or spectrophotometry.

One or more macromolecules can form the bulk (such as at least 40% by weight and up to, typically less than, 100%, for example 50%, 60%, 70%, 80%, 90%, 95%, or any ranges there between) of the microparticle. One or more active agents (such as organic or inorganic natural or synthetic pharmaceutical compounds or drugs, which may or may not be capable of having a tertiary and quaternary structure) can be covalently and/or non-covalently associated with at least a portion (such as the portion present on the surface of the microparticle) or substantially all of the one or more macromolecules (e.g., carriers, affinity molecules) and/or the one or more polymers (e.g., as complexes or conjugates thereof). It will be understood by those skilled in the art that the macromolecule can be a portion of a larger macromolecule. It will be further understood that an affinity molecule can be either the receptor portion or the ligand portion of a receptor-ligand interaction. Examples of ligands that interact with other biomolecules include viruses, bacteria, polysaccharides, or toxins that act as antigens to generate an immune response when administered to an animal and cause the production of antibodies.

Non-limiting examples of macromolecules include those disclosed in U.S. Pat. No. 6,458,387, columns 12-16, the entirety of which is incorporated herein by express reference thereto. In one example, the macromolecule can have a moderate molecular weight of 10 kD to 100 kD, such as 30 kD to 80 kD. In another example, the macromolecule can have a net electric charge that is negative (e.g., anionic or acidic), such as −5 or less, or −10 or less. In a further example, the macromolecule can have an isoelectric point pI that is greater than 7, or 7 or less, such as 6 or less, or 5 or less. In a further example, the macromolecule is anionic, such as polyanionic. In a further example, the macromolecule has one or more of the same or different anionic functional groups. In a further example, the macromolecule can have 30 or more of the same or different (e.g., anionic and/or cationic) functional groups (e.g., carboxyl groups, amine groups, thiol groups), such as 50 or more of one or more of such groups, or 70 or more of one or more of such groups. In a further example, the macromolecule can be non-immunogenic. Suitable examples of such macromolecules include, without limitation, albumins such as human serum albumin (HSA), bovine serum albumin (BSA), ovalbumin, and α-lactalbumin.

Non-limiting examples of polymers including ionic polymers such as: anionic, particularly polyanionic, polymers (e.g., polyanionic carbohydrates such as polyanionic polysaccharides including polysaccharide sulfates) such as dextran sulfate, heparin sulfate, heparan sulfate, chondroitin sulfate, galacturonic acids, alginates, mannuronic acid, guluronic acid, hyaluronic acid, heparin, chitin, chitosan, glycosaminoglycans, proteoglycans polystyrene sulfate, carboxymethylcellulose, polyaspartic acid, polyglutamic acid, polyacrylates, polycyanoacrylates, polyacetates, poly-β-hydroxybutyrates, polyvinylpyrrolidone, polyanionic dendrimers); cationic, particularly polycationic, polymers such as polylysine, polyarginine, protamine, protamine sulfate, polycitrulline, polyornithine, polyimino acids, polyiminotyrosine, cholestyramine resin, diethylaminoethylcellulose, histones, myelinbasic protein, polymyxin B sulfate, bradykinin, spermine, putrescine, octylarginine, polycationic dendrimers); and non-ionic and mixed ionic and non-ionic polymers (e.g., albumins such as HSA and BSA, IgG, IgM, hydrophobic polymers, non-ionic hydrophilic polymers, silicone, zein, lignin, surfactants, fatty acids, phospholipids, gelatins); Also useful in the composition and/or the preparation of the microparticles of the present disclosure are non-polymeric organic and/or inorganic polyvalent cations, such as, without limitation, metal cations (e.g., Zn²⁺, Mg²⁺, Ca²⁺, Cu^{2+ to 4+}, Fe^{2+ to 3+}, Be²⁺, Sr²⁺, Ba²⁺, Mn²⁺, Co^{2+ to 6+}, Ni^{2+to 6+}, Cd²⁺, Mo²⁺, Ra²⁺, Al³⁺, Tb³⁺, Y³⁺).

Non-limiting examples of active agents include those disclosed in U.S. Pat. No. 6,458,387, columns 20-23, the entirety of which is incorporated herein by express reference thereto. In one example, the active agent can be substantially or essentially free of functional carboxyl groups and primary amine groups. In another example, the active agent can have one or more of the same or different nucleophilic groups, such as one or more hydroxyl groups. In a further example, the active agent can have one or more of the same or different amino acid residues chosen from serine, threonine, and tyrosine. In a further example, the active agent can have one or more of the same or different oligopeptides segments (such as tripeptide segments) Z₁-Z₂-Z₃, in which Z₁ is a histidine residue, Z₂ is different from Z₁ and Z₃, Z₂ being a single amino acid residue or a chain of two or more amino acid residues, and Z₃ is a serine residue or threonine residue (or a tyrosine residue). Non-limiting examples of active agent having at least one such tripeptide segment include LHRH agonists, LHRH antagonists, LHRH analogs, such as leuprolide, goserelin, buserelin, gonadorelin, histrelin, nafarelin, deslorelin, fertirelin, triptorelin, salts thereof (e.g., acetate, trifluoroacetate, hydrazide, amide, hydrochloride, thereof), such as leuprolide acetate and leuprolide hydrochloride, and combinations of two or more thereof. At least some of these active agents are protonatable or protonated (partially or fully, for example, as in aqueous solution or in salt form).

Also associated with the microparticle, typically together with the one or more active agents, can include one or more non-polymeric polyanionic compounds. In one example, the non-polymeric polyanionic compound is allowed to be associated with the microparticle and/or the active agent during the process during which the active agent is allowed to be associated with the microparticle, like a co-agent during a crosslink reaction. The non-polymeric polyanionic compound can be capable of reducing or minimizing side reactions during the process through which the active agent is covalently and/or non-covalently associated with the one or more macromolecules and/or the one or more polymers. The non-polymeric polyanionic compound can be capable of facilitating the incorporation of the active agent into the microparticles. The non-polymeric polyanionic compound can be capable of modifying the release kinetics of the active agent from the microparticles, such as retarding or enhancing total release amount and/or release rate, increasing or decreasing the initial burst, lengthening or shortening the time period of sustained release phase, and/or otherwise altering the release profile. The non-polymeric polyanionic compound can be a relatively small molecule (i.e., not a polymer), having a relatively small number of the same or different functional groups (e.g., 8 or less, 5 or less, 2 or more, 3 or more). Non-limiting examples of the same or different functional groups include carboxyl, primary amine, secondary amine, phosphate, hydroxyl, hydroxide, and hydroxyamino groups. In one example, at least two of the same or different functional groups can be ionic or ionizable groups (e.g., anionic groups such as carboxyl, phosphate; cationic groups such as primary amine). In another example, the non-polymeric polyanionic compound can have different numbers of anionic groups and cationic groups (the difference being 1, 2, 3, or more), such as more anionic groups than cationic groups.

The non-polymeric polyanionic compound can be capable of forming an intermediate during the incorporation reaction as described above, and the intermediate is capable of having a net electric charge that is different (such as being different in sign and/or magnitude) from that of the non-polymeric polyanionic compound. In one example, the non-polymeric polyanionic compound can be capable of forming an intermediate in association with a crosslink activator as disclosed herein, and the intermediate can be neutral or cationic. In one example, the intermediate is incapable of cyclization or any other internal reactions that would consume one or more of the functional groups of the non-polymeric polyanionic compound. Non-limiting examples of suitable non-polymeric polyanionic compounds include non-polymeric compounds that contain two or more acid groups (like carboxyl groups) and being acidic, such as diacids (including dicarboxylic acids), triacids (including tricarboxylic acids), tetracids (including tetracarboxylic acids), anhydrides thereof, analogs thereof, salts thereof, and combinations of two or more thereof. The non-polymeric polyanionic compound can optionally further have one or more cationic groups and/or positively ionizable groups, such as amine groups, as in acidic polyanionic amino acids (such as monoamino diacids, monoamino triacids, diamino triacids), anhydrides thereof, analogs thereof, salts thereof, and combinations of two or more thereof. In a non-limiting example, the non-polymeric polyanionic compound includes one or more of aspartic acid, glutamic acid, salts thereof (like sodium salts thereof), and combinations of two or more thereof. In another non-limiting example, the non-polymeric polyanionic compound is aqueous-soluble, such as being water-soluble. In a further non-limiting example, the non-polymeric polyanionic compound is not a fatty acid, which includes fatty monoacids, fatty diacids, and other fatty polyacids.

The amount of the non-polymeric polyanionic compound incorporated into the microparticle can be such that it does not adversely affect desired characteristics of the microparticle (e.g., substantially spherical, high payload of active agent, substantially free of microparticle aggregation, monodisperse size distribution, controlled release of active agent). Percentage of the non-polymeric polyanionic compound by weight of the microparticle can be 5% or less, such as 3% or less, or 1% or less, but greater than 0%. Molar ratio of the non-polymeric polyanionic compound to the active agent (both of which are associated with the microparticle) can be 2:1 or less, such as 1:1 or less, or 1:2 or less. When a microparticle containing at least one non-polymeric polyanionic compound is compared to another microparticle that is free of the at least one non-polymeric polyanionic compound but is otherwise formed in a manner identical to the former microparticle, the former microparticle can have a release profile of the active agent the same as or different from that of the later microparticle. In one example, the release profile of the former microparticle has an initial burst that is greater than that of the later microparticle.

Methods of forming the microparticles of the present disclosure are not particularly limited, and include, for example, those disclosed in U.S. Pat. Nos. 6,458,387, 6,268,053, 5,849,884, and 5,578,709, the disclosures of which are incorporated herein in their entirety. In a non-limiting method, microparticles, such as microspheres, can be fabricated, optionally in the absence of additional oils or organic solvents, by combining and then mixing, in a flowable medium (such as an aqueous solution) one or more macromolecules and one or more polymers with one or more solubility-reducing agents. When a polyionic, such as polyanionic, polymer is used, a polyvalent cation or a salt thereof can be added, typically essentially simultaneously (within 30 minutes of the addition of the other ingredients), into the mixture as a solid or solution thereof. Optionally, additional stabilizing agents such as gelatin can be added as well. Optionally, one or more surfactants (e.g., carboxymethylcellulose, Tween®, Lutrol®, Pluronic®, Brij®, Span®, Emulsan®), such as those having a low viscosity of 100 cP or less, can also be added in the mixture, especially for forming uniform microspheres.

“Solubility-reducing agent” refers to a material that is capable of reducing the solubility of the macromolecule and/or the polymer in the solution, and facilitating the formation and/or stabilization of the microparticles. As understood by one skilled in the art, solubility-reducing agents can be aqueous-soluble and/or water-soluble, and can be ionic (e.g., polycationic, polyanionic) or non-ionic. Non-limiting examples of solubility-reducing agents include ionic polymers (e.g., polycationic, polyanionic) and non-ionic polymers, such as the water-soluble polymers disclosed in U.S. Pat. No. 6,458,387, columns 16-18, the entirety of which is incorporated herein by express reference thereto. Specific examples include starch (e.g., hetastarch, hydroxyethylstarch (HES), such as those with a molecular weight of 500 kD or greater, or 1,000 kD to 2,000 kD), polyvinylpyrrolidone (PVP), polyethylene glycol (PEG), dextran, and polyoxyethylene-polyoxypropylene copolymer (poloxomer). The polymers can be used singly or in combination of two or more thereof, such as in equal weight combinations thereof (e.g., PVP/PEG, PVP/hetastarch, PVP/HES).

Prior to mixing, the macromolecule and the polymer can be present in a first liquid phase, while the solubility-reducing agent can be present in a second liquid phase different from the first liquid phase. Alternatively, all materials can be present in a single continuous liquid phase. Percentage by weight of all solubility-reducing agent(s) in the total liquid mixture can be depends at least in part on the desired size of the microparticles: for microparticle size of 50 μm or greater, 20% or less; for microparticle size of less than 50 μm and greater than 10 μm, such as 15-35 μm, greater than 20% to less than 40%; for microparticle size of 10 μm or less, 40% or greater. Percentage by weight of each of the macromolecule and the polymer in the total liquid mixture can be 0.5 to 5%, such as 1% to 3%, or the combination is 3% to 5%. Weight ratio of the macromolecule to the polymer can be 1:10 or greater, typically 20:1 or less, such as 1:2 to 10:1, 1:1 to 5:1, or 1.5:1 to 3:1. Weight ratio of the solubility-reducing agent to the combination of the macromolecule and the polymer can be 1:1 or greater, typically 1,000:1 or less, such as 3:1 to 30:1, 4:1 to 16:1, 5:1 to 12:1, 7:1 to 9:1, or 8:1. Agitation means to mix the separated liquid phases into a single homogeneous continuous phase, such as an emulsion, include any and all of those know to one skilled in the art, such as sonication, vortexing, stirring, vibration, oscillation, and rocking, which can be used singly or in combination of two or more thereof. The mixing time can be between 5 minutes and 1 hour, such as between 10 minutes and 45 minutes, or between 15 minutes and 30 minutes. The mixing temperature can be at or below ambient temperature, but above the freezing temperature of the mixture, such as above 0° C. and below 30° C.

The pH of the aqueous solution can be adjusted, before, after or during agitation of the mixture, to one near the isoelectric point (pI) of the macromolecule. That is, the pH of the aqueous solution can be greater than, equal to, or less than the pI of the macromolecule, with the difference there between being 4 pH units or less, such as 3 pH units or less, 2 pH units or less, 1.5 pH units or less, or 1 pH unit or less. The pH adjustment can be made by adding to the macromolecule solution, the water-soluble polymer solution, and/or the mixture thereof an acid or base as a solid or solution, or a buffer or other pH-adjusting solution or solid salt in accordance with methods well known to those skilled in the art. In one example, the water-soluble polymer is dissolved in a buffer having a pH near the pI of the macromolecule as described above to form a solution, which is then mixed with another aqueous solution containing the macromolecule, resulting in an aqueous mixture with a pH near the pI of the macromolecule as described above.

Although not wishing to be bound to any particular theory or mechanism, it is believed that the microparticles formed during mixing process described above can be further processed to stabilize them into discrete, solid, and amorphous microparticles. Non-limiting examples of such stabilizing processes include subjecting the mixture to a change in one or more conditions (e.g., temperature, pH, mixture composition), such as one or more energy sources (e.g., heat, radiation, ionization), and/or addition of one or more crosslink agents, optionally in combination with agitation (e.g., sonication, vortexing, mixing, stirring, vibration, oscillation, rocking), and incubating the mixture for a predetermined period of time. The resulting microparticles are then separated from any unincorporated components present in the solution by physical separation methods (e.g., centrifugation, filtration, dialysis, diafiltration) known to one skilled in the art and can optionally be washed one or more times.

Non-limiting examples of crosslinking agents include dialdehydes, amines, multivalent ions, multifunctional molecules having an affinity for specific functional groups on the macromolecule being crosslinked, N-substituted maleimides, bifunctional alkyl halides, aryl halides, isocyanates, aliphatic or aromatic dicarboxylic acids, aliphatic or aromatic disulphonic acids, bifunctional imidoesters, and vinylsulphones. Additional crosslinking agents and methods for using same to stabilize a microsphere are described in U.S. Pat. No. 5,578,709, the entirety of which is incorporated herein by reference.

The temperature at which heating is used to stabilize the microparticles can be greater than ambient temperature, typically less than the boiling temperature of the mixture, such as at or greater than a thermal denaturation temperature of the macromolecule (that is, a temperature at or above which the native tertiary and/or quaternary structure of the macromolecule changes to a relatively more flexible one through the weakening and/or breaking of one or more associations such as bonds therein, and the macromolecule partially or fully unfolds and/or uncoils). Macromolecules of the present disclosure can have one or more thermal denature temperatures. For example, three thermal denaturation temperatures of 68° C., 85° C., and 120° C. have been observed in albumins such as human serum albumin. The incubation temperature can be between 37° C. and 150° C., such as 50° C. to 120° C., 70° C. to 100° C., or 85° C. to 90° C. The length of incubation is not particularly limited, and can be dependent at least in part upon the respective concentrations of water-soluble polymer and the macromolecule, as well as the energy level of the energy source and/or the concentration of the crosslink activator. The incubation time is typically between 5 minutes to 24 hours, such as between 30 minutes and 60 minutes.

One or more active agents can be incorporated in the microparticles by introducing the active agents to the flowable medium prior to, during, and/or after one or more stages of the microparticle formation as described above, such as before or during mixing the macromolecule with the solubility-reducing agent, before subjecting the mixture to the energy source and/or the crosslink activator, and/or after the microparticles are formed. The active agent can form covalent and/or non-covalent associations with one or more of the ingredients incorporated into the microparticle, such as the macromolecule and/or the polymer. The association can be formed before, during, and/or after the formation of the microparticle. In one example, the active agent is capable of being covalently associated with the macromolecule and/or the polymer in the presence of a crosslink activator (such as those that are aqueous-soluble and/or water-soluble) and, optionally, a co-agent such as a non-polymeric polyanionic compound. Non-limiting examples of crosslink activators include carbodiimides and salts thereof, such as 1-ethyl-3-(3-dimethylaminopropyl)carbodiimide hydrochloride (EDC), 1-ethyl-3-(4-azonia-4,4-dimethylpentyl)carbodiimide (EAC), and 1-cyclohexyl-3-(2-morpholinoethyl)carbodiimide metho-p-toluenesulfonate (CHMC), used singly or in combination of two or more thereof. The co-agent (such as one or more non-polymeric polyanionic compounds) can be added to the reaction mixture together with or after the active agent, while the crosslink activator is typically added last, after the co-agent.

Microparticles of the present disclosure can optionally be coated with one or more materials on its outermost surface. Materials and methods of coating the microparticles include, without limitation, those disclosed in U.S. Pat. No. 6,458,387, the entirety of which is incorporated herein by express reference thereto.

In one example, at least one macromolecule, such as human serum albumin, at least one polymer, such as dextran sulfate, an optional divalent cation source, such as calcium chloride, at least one solubility-reducing agent, such as hetastarch, and an optional surfactant, such as carboxymethylcellulose, can be combined to form a two-liquid-phase aqueous solution. Under continuous agitation, the solution can be allowed to form a continuous aqueous phase, heated to a first temperature at or above a thermal denaturation temperature of the macromolecule, such as 87° C., incubated for a predetermined period of time, then cooled to a second temperature below the lowest thermal denaturation temperature of the macromolecule, such as ambient temperature (like 25° C. to 40° C.), and incubated for another predetermined period of time, to fabricate the preformed microparticles. The preformed microparticles can be collected by any known separation means, such as centrifugation, and used as is, or optionally washed one or more time, typically with fresh volumes of the reaction buffer, followed by solutions of stabilizing agents (aqueous solutions containing divalent cations such as 5% w/v CaCl2 in deionized water) and pure deionized water, if desired.

Following optional washing, the preformed microparticles can be used in the form of a solid pellet, or re-suspended in a reaction medium, such as an aqueous buffer at a non-neutral pH (like one that differs from a neutral pH by at least 0.5 pH units, such as 6.5 or less or 7.5 or greater; or by at least 1 pH unit, such as 6.0 or less or 8.0 or greater). The active agent, such as a LHRH agonist, a LHRH antagonist, a LHRH analog, or a combination of two or more thereof, in a predetermined amount, such as 5% to 100% by weight of the macromolecule in the preformed microparticle, or 10% to 50%, or 20% to 40%, or 30% to 35%, can be dissolved in a volume of the reaction medium and mixed with the microsphere pellet or the re-suspension to form a re-suspension mixture, which can optionally be incubated under continuous agitation at a third temperature of 45° C. or less, such as 40° C. to greater than the freezing point of the suspension, or ambient temperature (such as 37° C. to 25° C.) or lower, for 10 minutes to 12 hours, such as 15 minutes to 6 hours, or 20 minutes to 1 hour. The preformed microparticle can be associated (covalently, electrostatically, and/or otherwise) with the active agent during and/or following this incubation. The non-polymeric polyanionic compound (e.g., aspartic acid, glutamic acid, salts thereof, and combinations of two or more thereof), in a predetermined amount, such as with a molar ratio to the total amount of active agent added of 20:1 or less (e.g., 10:1 or less, 1:1 or greater, 3:1 or greater), can be dissolved in a volume of the reaction medium and mixed into the mixture, and optionally further incubated for 1 minute or longer, such as 1 hours or less, or 15 minutes or less, under continuous agitation at the third temperature. The preformed microparticle can be associated (covalently, electrostatically, and/or otherwise) with the non-polymeric polyanionic compound during and/or following this incubation. The active agent can be associated (covalently, electrostatically, and/or otherwise) with the non-polymeric polyanionic compound during and/or following this incubation.

Then the re-suspension mixture can be heated or cooled to a fourth temperature of 60° C. or lower, which can be the same as or different from the third temperature, such as 45° C. or lower, ambient temperature (such as 37° C. to 25° C.) or lower, 2° C. or higher, or 10° C. or higher. The crosslink activator, such as EDC, in a predetermined amount, such as 20% to 200% by weight of the macromolecule in the preformed microparticle (e.g., 40% to 100%, 50% to 80%, 57%), and/or with a molar ratio to the total amount of the active agent added of 30:1 or less (e.g., 25:1 to 1:1, 20:1 to 5:1, 15:1 to 8:1), can be dissolved in a volume of the reaction medium and mixed slowly into the mixture, and incubated under continuous agitation at the fourth temperature for a predetermined period of 10 minutes to 10 hours (e.g., 1 hour to 5 hours, 3 hours). The crosslink activator can be added in two or more portions of the same or different amounts at different time points during the reaction incubation period (at least one portion must be added at the beginning of the reaction incubation) or added in its entirety at the beginning of the reaction incubation.

Upon concluding the reaction incubation, the temperature of the reaction medium can be returned to ambient temperature (such as 25° C. or lower), the resulting microparticles can be harvested through separation and optionally washing, such as one or more (e.g., 3, 4, 5, or more, or continuous) centrifugal washings with deionized water and/or fresh volumes of the reaction buffer, followed by one or more or continuous centrifugal washings (optionally at a fifth elevated temperature such as 35° C. to 40° C.) with one or more solutions containing one or more divalent cations (such as divalent cation solutions disclosed herein), and by one or more washings with ambient temperature (such as about 25° C.) deionized water.

The microparticles described herein are useful for a wide variety of separations, diagnostic, therapeutic, industrial, commercial, cosmetic, and research purposes or for any purpose requiring the incorporation of and stabilization of an active molecule, reactant or drug. Thus, the microparticles of the invention are useful for medical and diagnostic applications, such as drug delivery, vaccination, gene therapy and histopathological or in vivo tissue or tumor imaging. Accordingly, the microspheres are suitable for oral or parenteral administration; mucosal administration; ophthalmic administration; intravenous, subcutaneous, intra articular, or intramuscular injection; administration by inhalation; and topical administration.

The Examples described below are intended to be illustrative of aspects, features and/or advantages of the invention. The Examples are not to be considered limiting or otherwise restrictive of the invention.

### EXAMPLE 1

In this Example, the effects of incorporation of non-polymeric polyanionic compounds on leuprolide release from microparticles were evaluated. Three Formulations A, B and C were used to form microspheres. Table I lists quantities of respective ingredients added into the reaction mixtures to form the microspheres. The reactions of associating leuprolide acetate and non-polymeric polyanionic compound with the preformed microparticles were performed in a reaction buffer (25 mM MES buffer [(2-morpholino)ethanesulfonic acid] at pH 6.0). The preformed microparticles were formed from a homogeneous mixture of human serum albumin (HSA, 70% to 80% by weight of the microparticles) and dextran sulfate (20% to 30% by weight of the microparticles). Leuprolide acetate (12 mg dissolved in 0.6 ml MES buffer) was mixed under agitation into suspension of the preformed microparticles at ambient temperature, with an HSA:leuprolide (w/w) ratio of 35:12. At ambient temperature, the non-polymeric polyanionic compound (13.3 mg aspartic acid or 14.7 mg glutamic acid dissolved in 0.2 ml MES buffer) was then mixed into the suspension mixture for Formulations A and B. The control (Formulation C) had a 0.2 ml fresh MES buffer (free of non-polymeric polyanionic compound) mixed into the suspension mixture.

The suspension mixtures were heated in an oven at an incubation temperature of 37° C., and incubated for 3 hours, during which three portions of EDC (100 mg/ml in MES buffer) was mixed into each suspension mixture: 0.3 ml at the beginning of the reaction incubation, 0.1 ml at time 1.5 hours into the incubation, and 0.1 ml at time 2.5 hours into the incubation. After the 3-hour reaction incubation period, the heating was removed, and the resulting microspheres were separated from the liquid reaction medium by centrifugation, and then washed three times by centrifugation, each with fresh 1-ml aliquots of MES buffer. For each Formulation, the four supernatants (including the liquid reaction medium and the buffer washings) were combined into a single volume (i.e., the MES wash in Table I) of about 4.5 ml. The buffer-washed microspheres were further washed four times by centrifugation, each with 1 ml of a 5% (w/v) calcium chloride solution and once with 1 ml of deionized water. The five supernatants were combined into a single volume (i.e., the CaCl₂ wash in Table I) of about 5 ml. Leuprolide contents in the MES wash and the CaCl₂ wash were measured with uv spectrophotometry (absorbance at 280 nm) to calculate leuprolide contents in the microspheres. The microspheres of all three Formulations A, B, and C retained comparable amounts of leuprolide added (i.e., yield).

TABLE I

| | Formulation | | | | | |
|---|---|---|---|---|---|---|
| | A | | B | | C | |
| Microparticles | 50 mg (35 mg) | | 50 mg (35 mg) | | 50 mg (35 mg) | |
| (HSA content) | | | | | | |
| Leuprolide acetate | 12 | mg | 12 | mg | 12 | mg |
| Aspartic acid | 13.3 | mg | 0 | | 0 | |
| Glutamic acid | 0 | | 14.7 | mg0 | 0 | |
| EDC (at 0 hour and | 30 | mg | 30 | mg | 30 | mg |
| 37° C.) | | | | | | |
| EDC (at 1.5 hours and | 10 | mg | 10 | mg | 10 | mg |
| 37° C.) | | | | | | |
| EDC (at 2.5 hours and | 10 | mg | 10 | mg | 10 | mg |
| 37° C.) | | | | | | |
| Leuprolide Content | | | | | | |
| MES wash | 2.0 | mg | 1.65 | mg | 1.28 | mg |
| CaCl₂ wash | 1.25 | mg | 1.67 | mg | 1.62 | mg |
| Microspheres | 8.75 | mg | 8.68 | mg | 9.1 | mg |

| | | | |
|---|---|---|---|
| Yield % | 72.9% | 72.3% | 75.8% |

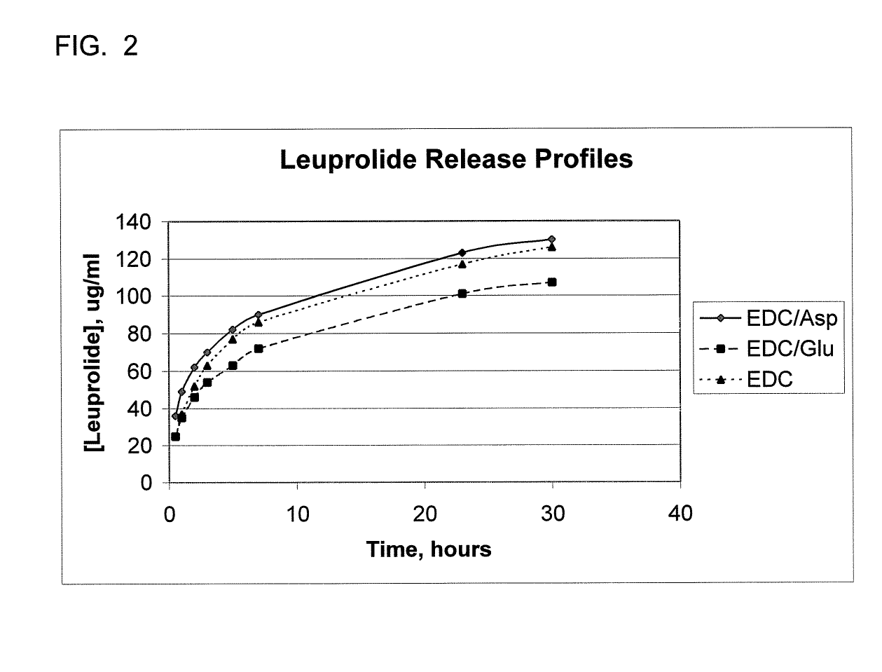

The thoroughly washed microspheres were then incubated with agitation in a release buffer (50 mM Na phosphate, 0.5 M sodium chloride, and 0.05% Tween 80, pH 7.5) at 37° C. (in an oven) for 3 hours to release leuprolide. Aliquots of the release medium were taken at various time intervals and centrifuged to collect the supernatants for measurement of leuprolide content therein by UV spectrometry (absorbance at 280 nm). As shown in FIG. 2, in which the amount of leuprolide released into the release medium as a function of time was plotted, the incorporation of the non-polymeric polyanionic compound into the microspheres as described herein is capable of changing the leuprolide release profile there from. In particular, the incorporation of aspartic acid provided a greater initial burst than that of the control, while the subsequent sustained release phase was substantially the same. The incorporation of glutamic acid provided an initial burst comparable to that of the control, while the rate of release during the subsequent sustained release phase was markedly reduced.

### EXAMPLE 2

In this example, the incorporation of aspartic acid into the microspheres was verified using [14]C-labeled aspartic acid. Aspartic acid solution (0.50 M) was prepared in MES buffer as described in Example 1. To a 0.20 ml aliquot of the above solution was mixed in 5 microliters of [14]C-labeled aspartic acid in the same MES buffer, a 60-microliter aliquot of the mixture was taken for scintillation counting, and another 60-microliter aliquot was used in the microsphere preparation below.

Two mixtures each containing microspheres (50 mg, same as in Example 1, formed from HSA and dextran sulfate as disclosed herein) and leuprolide hydrochloride (12 mg, 9.6 micromole) in 0.60 ml MES buffer as in Example 1 were formed. The mixtures were kept in suspension on a rotator at ambient temperature for 20 minutes. Mixed in one mixture was 60 microliters of aspartic acid (30 micromoles, free of [14]C-labeled aspartic acid), and in the other mixture was 60 microliters of the solution containing [14]C-labeled aspartic acid, as described above. Then 16 mg of EDC hydrochloride (83 micromoles) dissolved in 0.34 ml of MES buffer was charged into each mixture in one aliquot. The two mixtures were then incubated at 37° C. (in an oven) with agitation (on the rotator) for three hours. Each mixture was centrifuged to remove the liquid reaction medium, and then centrifugally washed sequentially once with 1 ml fresh MES buffer, five times with 1 ml calcium chloride (5% w/v) to give a combined 2 ml MES wash, a combined 5 ml CaCl₂ wash, and a pellet of leuprolide-loaded microspheres.

The samples (the MES wash, the CaCl₂ wash, and the microspheres) containing [14]C-labeled aspartic acid were analyzed using a scintillation counter and yielded a 101% recovery of total [14]C-labeled aspartic acid added. The samples that are free of [14]C-labeled aspartic acid were analyzed using a UV-spectrometer to calculate the amount of leuprolide incorporated into the microspheres. The results are summarized below in Table II. The molar ratio of incorporated aspartic acid (3 micromoles) to incorporated leuprolide (6.52 micromoles) in the microspheres was estimated to be about 0.46.

TABLE II

| | [14]C Asp | | Leuprolide | |
|---|---|---|---|---|
| | % of | | % of | |
| | total added | micromoles | total added | Micromoles |
| MES wash | 89% | 26.7 | 8.70% | 0.83 |
| CaCl2 wash | 1% | 0.3 | 23.40% | 2.25 |
| Microsphere | 10% | 3 | 67.90% | 6.52 |

### EXAMPLE 3

In a further example, the extent of modification of leuprolide release kinetics due to varying molar ratio of leuprolide:(aspartic acid):EDC added into the reaction mixture during the formation of the microspheres was evaluated. Microspheres were prepared as described in Example 1 above, using five different formulations of varying molar ratios listed in Table III below, except that leuprolide hydrochloride was used in place of leuprolide acetate, and EDC was added in one aliquot. After the reaction incubation, the microspheres were washed five times with fresh MES buffer, five times with a 5% (w/v) calcium chloride solution, and at least once with deionized water. The thoroughly washed microspheres were then incubated in the release buffer of Example 1 and aliquots removed at regular time intervals to determine leuprolide release kinetics, as described in Example 1 above.

TABLE III

| | Molar Ratio of |
|---|---|
| Formulations | (Leuprolide):(Aspartic acid):(EDC) |
| # 1 | 10:100:156 |
| # 2 | 10:60:104 |
| # 3 | 10:30:84 |
| # 4 | 10:0:156 |
| # 5 | 10:0:220 |
| Manufacturing Scale | |

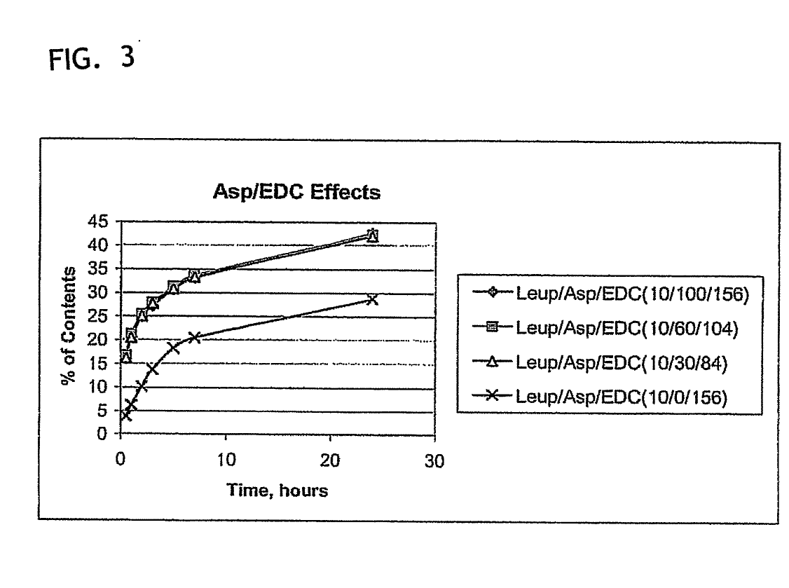

As shown in FIG. 3, the leuprolide release profiles of microspheres of Formulations #1, #2, and #3 (all containing aspartic acid) were substantially similar, indicating little correlation between the leuprolide release kinetics and the molar ratio of leuprolide:(aspartic acid):EDC used during the formation of the microspheres. The microspheres containing aspartic acid consistently displayed an increased initial burst in leuprolide release kinetics as compared to microspheres without aspartic acid.

### EXAMPLE 4

In a further example, scale-up preparations of leuprolide-containing microspheres were fabricated and extended release kinetics of leuprolide in an acidic environment was evaluated. In this example, microspheres of Formulations A, B, C, or D (Table IV) were prepared on a relatively large scale (40 ml reaction volume). Table IV lists quantities of respective ingredients added into the reaction mixtures to form the microspheres. General protocols described in Examples 1 and 3 were followed, except that EDC HCl was added in two different portions (one at beginning of reaction incubation, another at 2 hours into the incubation) in Formulations A and D.

TABLE IV

| | Formulations | | | | |
|---|---|---|---|---|---|
| | A | B | C | D | E |
| Microparticles | 2.0 g | 2.0 g | 2.0 g | 2.0 g | 2.0 g |
| Leuprolide | 480 mg | | | | 480 mg |
| Acetate | (0.378 mmol) | | | | (0.378 mmol) |
| Leuprolide HCI | | 480 mg | 480 mg | 480 mg | |
| | | (0.385 mmol) | (0.385 mmol) | (0.385 mmol) | |
| Aspartic Acid | 4.0 mmol | 2.4 mmol | 1.2 mmol | 1.2 mmol | |
| EDC HCI (at 0 | 1.2 g | 0.80 g | 0.64 g | 0.36 g | 1.2 g |
| hour) | (6.26 mmol) | (4.17 mmol) | (3.34 mmol) | (1.88 mmol) | (6.26 mmol) |
| EDC HCI (at 2 | 0.4 g | 0 | 0 | 0.28 g | 0.4 g |
| hours) | (2.09 mmol) | | | (1.46 mmol) | (2.09 mmol) |

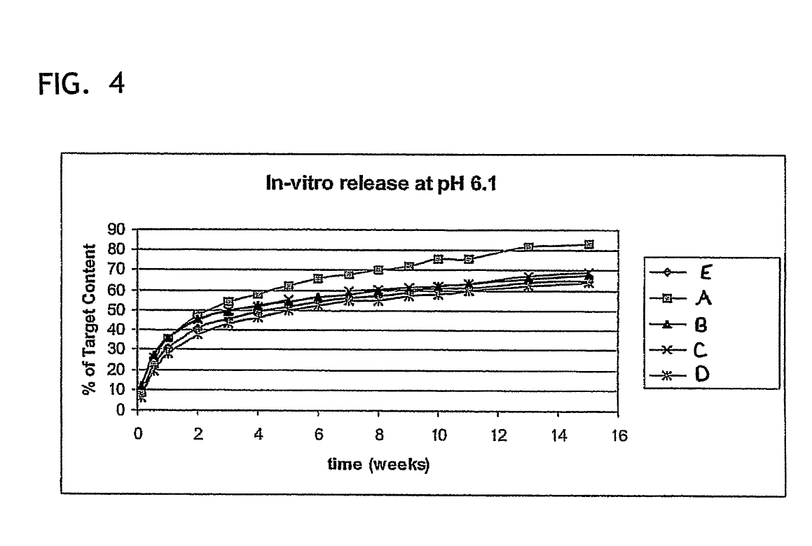

The resulting microspheres were assayed for their release kinetics over an extended period in a release buffer (same composition as in Example 1, but at pH 6.1), as described in Example 1. Microspheres formed from a reference formulation (Formulation E, with leuprolide acetate and without aspartic acid) was also assayed for extended release. As shown in FIG. 4, the leuprolide release kinetics of microspheres of Formulation A appears to differ from those of the other formulations.

It will be understood that the examples of the present disclosure are illustrative of some of the applications of the principles of the present invention. Numerous modifications can be made by those skilled in the art without departing from the true spirit and scope of the disclosure. Various elements disclosed herein can be used in any combination and are not limited to procure combinations that are specifically outlined herein.

1. A method for preparing a microparticle, comprising:
providing a preformed microparticle;
exposing the preformed microparticle to at least one active agent and at least one non-polymeric polyanionic compound; and
forming the microparticle comprising the preformed microparticle in association with the at least one active agent and the at least one non-polymeric polyanionic compound.

2. The method of claim 1, wherein the preformed microparticle is provided as a suspension in a flowable medium.

3. The method of claim 2, wherein the exposing comprises mixing, together or separately, the at least one active agent and the at least one non-polymeric polyanionic compound into the suspension.

4. The method of claim 1, wherein the at least one non-polymeric polyanionic compound comprises at least one of diacids, triacids, tetracids, polyanionic amino acids, anhydrides thereof, analogs thereof, salts thereof, and combinations of two or more thereof.

5. The method of claim 1, wherein the salt of the at least one non-polymeric polyanionic compound is selected from the group consisting of salts having monovalent metal cations, divalent metal cations, polyvalent metal cations, organic cations, and combinations of two or more thereof.

6. The method of claim 1, wherein the at least one non-polymeric polyanionic compound comprises at least one of aspartic acid, glutamic acid, salts thereof, and combinations of two or more thereof.

7. The method of claim 1, wherein the at least one active agent is free of primary amine groups and carboxyl groups, and comprises at least one nucleophilic group.

8. The method of claim 1, wherein the at least one active agent is protonatable or protonated.

9. The method of claim 1, wherein the at least one active agent is in association with the at least one non-polymeric polyanionic compound.

10. The method of claim 1, wherein the at least one active agent comprises at least one of LHRH, agonists thereof, antagonists thereof, analogs thereof, salts thereof, and combinations of two or more thereof.

11. The method of claim 1, further comprising forming a mixture comprising the preformed microparticle, the at least one active agent, and the at least one non-polymeric polyanionic compound, and exposing the mixture to at least one crosslink activator.

12. The method of claim 11, wherein the at least one crosslink activator comprises at least one of carbodiimides and salts thereof.

13. The method of claim 11, wherein the at least one crosslink activator comprises at least one of 1-ethyl-3-(3-dimethylaminopropyl)carbodiimide, 1-ethyl-3-(4-azonia-4,4-dimethylpentyl)carbodiimide, 1-cyclohexyl-3-(2-morpholinoethyl)carbodiimide, salts thereof, and combinations of two or more thereof.

14. The method of claim 11, wherein the mixture is exposed to the at least one crosslink activator at a temperature between 10° C. and 60° C., and at a pH of 6.5 or lower.

15. The method of claim 1, wherein the preformed microparticle comprises a homogeneous mixture comprising at least one anionic macromolecule and at least one anionic polymer.

16. A microparticle comprising:
at least one anionic macromolecule;
at least one anionic polymer;
at least one active agent; and
at least one non-polymeric polyanionic compound, wherein the at least one anionic macromolecule and the at least one anionic polymer are homogeneously distributed with each other.

17. The method of claim 16, wherein the at least one non-polymeric polyanionic compound comprises at least one of diacids, triacids, tetracids, polyanionic amino acids, anhydrides thereof, analogs thereof, salts thereof, and combinations of two or more thereof.

18. The method of claim 17, wherein the salt of the at least one non-polymeric polyanionic compound is selected from the group consisting of salts having monovalent metal cations, divalent metal cations, polyvalent metal cations, organic cations, and combinations of two or more thereof.

19. The method of claim 16, wherein the at least one non-polymeric polyanionic compound comprises at least one of aspartic acid, glutamic acid, salts thereof, and combinations of two or more thereof.

20. The microparticle of claim 16, wherein the at least one anionic macromolecule comprises human serum albumin, and the at least one anionic polymer comprises dextran sulfate.

21. The microparticle of claim 16, wherein the at least one non-polymeric polyanionic compound is aqueous-soluble.

22. The microparticle of claim 16, wherein the at least one non-polymeric polyanionic compound is not a fatty acid.

23. The microparticle of claim 16, wherein the at least one active agent is protonatable or protonated.

24. The microparticle of claim 16, wherein the at least one active agent is in association with the at least one non-polymeric polyanionic compound.

25. The microparticle of claim 16, wherein the at least one active agent is free of primary amine groups and carboxyl groups, and comprises at least one nucleophilic group.

26. The microparticle of claim 23, wherein the nucleophilic group is a hydroxyl group.

27. The microparticle of claim 16, wherein the at least one active agent comprises at least one histidine residue and at least one of serine residue, threonine residue, tyrosine residue, and combinations of two or more thereof.

28. The microparticle of claim 16, wherein the at least one active agent comprises at least one of the same or different oligopeptide segments each having the structure Z₁-Z₂-Z₃, where Z₁ is histidine residue, Z₂ is different from Z₁ and Z₃, Z₂ being a single amino acid residue or a chain of two or more amino acid residues, and Z₃ is serine residue or threonine residue.

29. The microparticle of claim 16, wherein the active agent comprises at least one of LHRH, agonists thereof, antagonists thereof, analogs thereof, salts thereof, and combinations of two or more thereof.

30. The microparticle of claim 16, wherein the microparticle comprises a core formed from the at least one anionic macromolecule and the at least one anionic polymer, and wherein the core is in association with the at least one active agent and the at least one non-polymeric polyanionic compound.

31. The microparticle of claim 16, wherein the active agent comprises at least one of leuprolide; goserelin; buserelin; gonadorelin; histrelin; nafarelin; deslorelin; fertirelin; triptorelin; and salts thereof selected from the group consisting of acetate, trifluoroacetate, hydrazide, amide, and hydrochloride; and combinations of two or more thereof.

32. A microparticle comprising:
at least one anionic macromolecule;
at least one anionic polymer;
at least one active agent selected from the group consisting of LHRH, agonists thereof, antagonists thereof, analogs thereof, salts thereof, and combinations of two or more thereof; and
at least one non-polymeric polyanionic compound selected from the group consisting of polyanionic amino acids, anhydrides thereof, analogs thereof, salts thereof, and combinations of two or more thereof.

33. The microparticle of claim 32, wherein the at least one active agent is selected from the group consisting of leuprolide, goserelin, buserelin, gonadorelin, histrelin, nafarelin, deslorelin, fertirelin, triptorelin, salts thereof, and combinations of two or more thereof; and the non-polymeric polyanionic compound is selected from the group consisting of aspartic acid, glutamic acid, and salts thereof.
